(19) United States
(12) Patent Application Publication
(10) Pub. No.: US 2009/0297602 A1
(43) Pub. Date: Dec. 3, 2009

(54) Modified Release Loxoprofen Compositions

(76) Inventors: John G. Devane, County Westmeath (IE); Paul Stark, County Westmeath (IE); Niall M.N. Fanning, County Westmeath (IE); Grvinder Singh Rekhi, Suwanee, GA (US); Gary Liversidge, Westchester, PA (US); Scott Jenkins, Downington, PA (US)

Correspondence Address: Fox Rothschild, LLP;Elan Pharma International Limited, 2000 Market Street, Philadelphia, PA, 19103, US

(21) Appl. No.: 11/568,925

(22) PCT Filed: May 9, 2006
(86) PCT No.: PCT/US06/17938

§ 371 Date: Jun. 16, 2009

Related U.S. Application Data
(63) Continuation-in-part of application No. 11/372,857, filed on Mar. 10, 2006.
Application No. 11/372,857 is a continuation-in-part of application No. 10/827,689, filed on Apr. 19, 2004.
Application No. 10/827,689 is a continuation of application No. 10/354,483, filed on Jan. 30, 2003, now Pat. No. 6,793,936.
Application No. 10/354,483 is a continuation of application No. 10/331,754, filed on Dec. 30, 2002, now Pat. No. 6,902,742.
Application No. 10/331,754 is a continuation of application No. 09/850,425, filed on May 7, 2001, now Pat. No. 6,730,325.
Application No. 09/850,425 is a continuation of application No. 09/566,636, filed on May 8, 2000, now Pat. No. 6,228,398.
Application No. 09/566,636 is a continuation of application No. PCT/US99/25632, filed on Nov. 1, 1999.
(60) Provisional application No. 60/679,517, filed on May 10, 2005.
(60) Provisional application No. 60/106,726, filed on Nov. 2, 1998.

(51) Int. Cl. A61K 9/10; A61K 9/16; A61K 31/216

(52) U.S. Cl. 424/484; 424/490; 424/497; 514/530

(57) ABSTRACT

The invention relates to a modified release composition comprising loxoprofen or a salt or derivative thereof that in operation delivers the drug in a pulsatile or continuous manner for the treatment of pain and/or inflammation. The composition may comprise a first loxoprofen component and one subsequent loxoprofen component, wherein the first loxoprofen component comprises an immediate release component and the subsequent loxoprofen component comprises a modified release component.

### CROSS-REFERENCE TO RELATED APPLICATIONS

This application claims the benefit of U.S. provisional Application No. 60/679,517, filed May 9, 2005 and U.S. application Ser. No. 11/372,857, filed Mar. 10, 2006, and is a continuation-in-part of U.S. application Ser. No. 11/372,857, filed Mar. 10, 2006, which is a continuation-in-part of U.S. application Ser. No. 10/827,689, filed Apr. 19, 2004, which is a continuation of U.S. application Ser. No. 10/354,483, filed Jan. 30, 2003, now U.S. Pat. No. 6,793,936, which in turn is a continuation of U.S. application Ser. No. 10/331,754, filed Dec. 30, 2002, now U.S. Pat. No. 6,902,742, which in turn is a continuation of U.S. application Ser. No. 09/850,425, filed May 7, 2001, now U.S. Pat. No. 6,730,325, which in turn is a continuation of U.S. application Ser. No. 09/566,636, filed May 8, 2000, now U.S. Pat. No. 6,228,398, which in turn is a continuation of PCT Application No. PCT/US99/25632, filed Nov. 1, 1999, which claims the benefit of U.S. provisional Application No. 60/106,726, filed Nov. 2, 1998.

### FIELD OF THE INVENTION

The present invention relates to novel compositions and dosage forms for the treatment of pain and inflammation. In particular, the present invention relates to novel compositions and dosage forms for the delivery of loxoprofen, or a salt or derivative thereof, and to methods of treatment using the same.

### BACKGROUND OF THE INVENTION

Loxoprofen is a phenylpropionic acid-type non-steroidal, anti-inflammatory drug (NSAID) prodrug. When orally administered, it is metabolized in the body (primarily in the liver and kidneys) into a trans-OH form, which exhibits potent anti-inflammatory activities. Loxoprofen is rapidity absorbed into the digestive tract localizing in the region of inflammation and serving as an anti-inflammatory analgesic for the management of pain and inflammation associated with skeletal muscle and joint disorders or resulting from operative procedures. Loxoprofen is particularly effective in the treatment of pain and inflammation resulting from chronic arthritis, rheumatism, lumbago, and trauma.

Loxoprofen appears as a white or slightly yellowish white crystalline powder. The chemical name of loxoprofen, also referred to as loxoprofen sodium, is monosodium 2-[4-(2-oxocyclopentylmethyl) phenyl] propanoate dehydrate. The empirical formula of loxoprofen is C₁₅H₁₇NaO₃.2H₂O and its molecular weight is 304.31. The structural formula of loxoprofen is shown below:

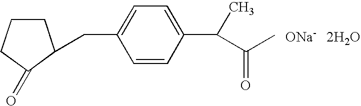

Loxoprofen is described, for example, in U.S. Pat. No. 6,248,350 for “External Formulation Containing Loxoprofen” which describes an anti-inflammatory analgesic patch containing loxoprofen and which is incorporated herein by reference in its entirety. Conventional loxoprofen tablets, such as LOXONIN® (marketed by Sankyo Co. Ltd. of Japan) are administered orally three times a day for the treatment of pain and inflammation.

Loxoprofen is of high therapeutic value for the treatment of pain and inflammation associated with skeletal muscle and joint disorders or resulting from operative procedures. Given that loxoprofen requires oral administration three times daily, strict patient compliance is a critical factor in the efficacy of loxoprofen. Moreover, such frequent administration often requires the attention of health care workers and contributes to the high costs associated with treatments involving loxoprofen. Thus, there is a need in the art for loxoprofen compositions which overcome these and other problems associated with the use of conventional forms of loxoprofen for the treatment of pain and inflammation.

The objective of many modified release drug formulations is to produce a substantially constant release of the drug compound. Indeed, it is often a specific object of these formulations to minimize the variation in plasma concentration levels associated with conventional frequent dosage regimes. Another objective of modified release drug formulations is to hasten the onset of action by minimizing to time from the administration of the drug to the achievement of a therapeutically effective plasma concentration. A further objective of modified release drug formulations is to maintain a therapeutically effective plasma concentration throughout the dosing interval. The achievement of two or all three of these objectives in conventional drug formulations, however, is problematic. Thus, modified release compositions or formulations which combine the benefits of at least two different release profiles to achieve a resultant plasma profile that minimizes variations in plasma concentration levels and/or provides rapid onset of action and/or maintains a therapeutically effective plasma concentration throughout the dosing interval is desirable.

Conventional frequent dosage regimes in which an immediate release (IR) dosage form is administered at periodic intervals typically give rise to a pulsatile plasma profile. In such cases, a peak in the plasma drug concentration is observed after administration of each IR dose with periods of lower drug concentration observed between consecutive administration time points. Such dosage regimes and the resultant pulsatile plasma profiles associated therewith can have particular pharmacological and therapeutic effects that are beneficial for certain drug therapies. For example, the wash out period provided by the drop in the plasma concentration of the active ingredient between peaks has been thought to be a contributing factor in reducing or preventing patient tolerance to various types of drugs.

For certain drugs, some of the therapeutic and pharmacological effects intrinsic in a pulsatile system may be lost or diminished as a result of the constant or nearly constant plasma concentration levels achieved by zero-order release drug delivery systems. Thus, modified release compositions or formulations which substantially mimic the release of frequent IR dosage regimes while reducing the need for frequent dosing is desirable.

A typical example of a drug which may produce tolerance in patients is methylphenidate. Methylphenidate, or α-phenyl-2-piperidine acetic acid methyl ester, is a stimulant affecting the central nervous and respiratory systems and is primarily used in the treatment of attention deficit hyperactivity disorder (ADHD). After absorption from the gastrointestinal tract (GIT), drug effects persist for 3-6 hours after oral administration of conventional IR tablets or up to about 8 hours after oral administration of extended release formulations. The total dosage is typically in the range of 5-30 mg per day, in exceptional cases rising to 60 mg/day. Under conventional dosage regimes, methylphenidate is given twice daily, typically with one dose given before breakfast and a second dose given before lunch. The last daily dose is preferably given several hours before retiring. Adverse effects associated with methylphenidate treatment include insomnia and the development of patient tolerance.

WO 98/14168 (Alza Corp.) teaches a dosage form and a method of administering methylphenidate in a sustained and constantly ascending rate. The dosage form disclosed comprises a plurality of beads comprising a hydrogel matrix with increasing amounts of the active ingredient therein, coated with varying amounts of a release rate controlling material. Appropriate combinations of the active ingredient dose and the number and thickness coating layers can be selected to give an ascending release profile in which the plasma concentration of the active ingredient continually increases over a given period of time. An object of WO 98/14168 is to release a dosage form at a constantly ascending rate specifically to avoid uneven blood levels (characterized by peaks and troughs) associated with conventional treatments using immediate release dosage formulations. As a result, this formulation does not deliver the active ingredient in either a pulsatile or a bimodal manner.

WO 97/03672 (Chiroscience Ltd.) discloses that methylphenidate exhibits a therapeutic effect when administered in the form of a racemic mixture or in the form of a single isomer (such as the RR d-threo enantiomer). Further, WO 97/03763 (Chiroscience Ltd.) discloses a sustained release formulation containing d-threo methylphenidate (dtmp). This disclosure teaches the use of a composition comprising a coating through which the dtmp passes in order to attain sustained release and achieve serum levels (of the active ingredient) of at least 50% cmax over a period of at least 8 hours. As above, this formulation does not deliver the active ingredient in either a pulsatile or a bimodal manner.

Shah et al., J. Cont. Rel. (1989) 9:169-175 purports to disclose that certain types of hydroxypropyl methylcellulose ethers compressed into a solid dosage form with a therapeutic agent may produce a bimodal release profile. However, it is noted that while polymers from one supplier yielded a bimodal profile, the same polymers with almost identical product specifications obtained from a different source gave non-bimodal release profiles.

Giunchedi et al., Int. J. Pharm (1991) 77:177-181 discloses the use of a hydrophilic matrix multiple-unit formulation for the pulsed release of ketoprofen. Giunchedi et al. teach that ketoprofen is rapidly eliminated from the blood after dosing (plasma half-life 1-3 hours) and consecutive pulses of drug may be more beneficial than constant release for some treatments. The multiple-unit formulation disclosed comprises four identical hydrophilic matrix tablets placed in a gelatin capsule. Although the in vivo studies show two peaks in the plasma profile there is no well defined wash out period and the variation between the peak and trough plasma levels is small.

Conte et al., Drug Dev. Ind. Pharm, (1989) 15:2583-2596 and EP 0 274 734 (Phamidea Srl) teach the use of a three layer tablet for delivery of ibuprofen in consecutive pulses. The three layer tablet is made up of a first layer containing the active ingredient, a barrier layer (the second layer) of semi-permeable material which is interposed between the first layer and a third layer containing an additional amount of active ingredient. The barrier layer and the third layer are housed in an impermeable casing. The first layer dissolves upon contact with a dissolving fluid while the third layer is only available after dissolution or rupture of the barrier layer. In such a tablet the first portion of active ingredient must be released instantly. This approach also requires the provision of a semi-permeable layer between the first and third layers in order to control the relative rates of delivery of the two portions of active ingredient. Additionally, rupture of the semi-permeable layer leads to uncontrolled dumping of the second portion of the active ingredient which may not be desirable.

U.S. Pat. No. 5,158,777 (E. R. Squibb & Sons Inc.) discloses a formulation comprising captopril within an enteric or delayed release coated pH stable core combined with additional captopril which is available for immediate release following administration. In order to form the pH stable core, chelating agents such as disodium edetate or surfactants such as polysorbate 80 are used either alone or in combination with a buffering agent. The compositions have an amount of captopril available for immediate release following oral administration and an additional amount of pH stabilized captopril available for release in the colon.

U.S. Pat. Nos. 4,728,512, 4,794,001 and 4,904,476 (American Home Products Corp.) relate to preparations providing three distinct releases. The preparation contains three groups of spheroids containing an active medicinal substance: the first group of spheroids is uncoated and rapidly disintegrates upon ingestion to release an initial dose of medicinal substance; the second group of spheroids is coated with a pH sensitive coat to provide a second dose; and the third group of spheroids is coated with a pH independent coat to provide to third dose. The preparation is designed to provide repeated release of medicinal substances which are extensively metabolized presystemically or have relatively short elimination half-lives.

U.S. Pat. No. 5,837,284 (Mehta et al) discloses a methylphenidate dosage form having immediate release and delayed release particles. The delayed release is provided by the use of ammonio methacrylate pH independent polymers combined with certain fillers.

### SUMMARY OF THE INVENTION

The present invention relates to compositions comprising a population of loxoprofen-containing particles, wherein the particles comprise a modified release coating, a modified release matrix material, or both, such that the composition delivers the loxoprofen, or a salt or derivative thereof, to a subject in a pulsatile or continuous manner following oral delivery of the composition to the subject. As used herein, the term “loxoprofen” includes the specific compound described above as well as all pharmaceutically acceptable salts and derivatives thereof. The present invention also relates to dosage forms comprising such compositions and methods for the treatment of pain and/or inflammation comprising the step of providing such dosage forms.

Other objects and advantages of the present invention will become readily apparent to those skilled in the art from the following detailed description, wherein the preferred embodiments of the invention are shown and described, simply by way of illustration of the best mode contemplated of carrying out the invention. As will be realized, the invention is capable of and encompasses other and different embodiments, and its several details are capable of modifications in various obvious respects, all without departing from the invention.

### BRIEF DESCRIPTION OF THE DRAWINGS

FIG. 1. Simulation of plasma concentrations obtained following dosing various percentages of CR (modified release) and IR (immediate release) loxoprofen.

### DETAILED DESCRIPTION OF THE INVENTION

The effectiveness of pharmaceutical compounds in the prevention and treatment of disease states depends on a variety of factors including the rate and duration of delivery of the compound from the dosage form to the patient. The combination of delivery rate and duration exhibited by a given dosage form in a patient can be described as its in vivo release profile and, depending on the pharmaceutical compound administered, will be associated with a concentration and duration of the pharmaceutical compound in the blood plasma, referred to as a plasma profile. As pharmaceutical compounds vary in their pharmacokinetic properties such as bioavailability, and rates of absorption and elimination, the release profile and the resultant plasma profile become important elements to consider in designing effective drug therapies.

The release profiles of dosage forms may exhibit different rates and durations of release and may be continuous or pulsatile. Continuous release profiles include release profiles in which a quantity of one or more pharmaceutical compounds is released continuously throughout the dosing interval at either a constant or variable rate. Pulsatile release profiles include release profiles in which at least two discrete quantities of one or more pharmaceutical compounds are released at different rates and/or over different time frames. For any given pharmaceutical compound or combination of such compounds, the release profile for a given dosage form gives rise to an associated plasma profile in a patient. When two or more components of a dosage form have different release profiles, the release profile of the dosage form as a whole is a combination of the individual release profiles and may be described generally as “multimodal.” The release profile of a two-component dosage form in which each component has a different release profile may described as “bimodal,” and the release profile of a three-component dosage form in which each component has a different release profile may described as “trimodal.”

Similar to the variables applicable to the release profile, the associated plasma profile in a patient may exhibit constant or variable blood plasma concentration levels of the pharmaceutical compounds over the duration of action and may be continuous or pulsatile. Continuous plasma profiles include plasma profiles of all rates and duration which exhibit a single plasma concentration maximum. Pulsatile plasma profiles include plasma profiles in which at least two higher blood plasma concentration levels of pharmaceutical compound are separated by a lower blood plasma concentration level and may be described generally as “multimodal.” Pulsatile plasma profiles exhibiting two peaks may be described as “bimodal” and plasma profiles exhibiting more than three peaks may be described as “trimodal.” Depending on, at least in part, the pharmacokinetics of the pharmaceutical compounds included in the dosage form as well as the release profiles of the individual components of the dosage form, a multimodal release profile may result in either a continuous or a pulsatile plasma profile upon administration to a patient.

In one embodiment the present invention provides a multiparticulate modified release composition which delivers loxoprofen in a pulsatile manner.

In another embodiment the present invention provides a multiparticulate modified release composition containing loxoprofen which produces a plasma profile substantially similar to the plasma profile produced by the administration of three IR dosage forms given sequentially.

In yet another embodiment the present invention provides a multiparticulate modified release composition which substantially mimics the pharmacological and therapeutic effects produced by the administration of three IR dosage forms of loxoprofen given sequentially.

In still another embodiment the present invention provides a multiparticulate modified release composition which delivers loxoprofen in a continuous manner.

In yet another embodiment the present invention provides a multiparticulate modified release composition in which a first portion of loxoprofen is released immediately upon administration and one or more subsequent portions of loxoprofen are released after an initial time delay.

In still another embodiment the present invention provides a multiparticulate modified release composition which provides rapid onset of action and a therapeutically effective plasma concentration level of loxoprofen for about 12 to about 24 hours.

In yet another embodiment the present invention provides solid oral dosage forms for once-daily or twice-daily administration comprising the multiparticulate modified release composition of the present invention.

In still another embodiment the present invention provides a method for the treatment of pain and/or inflammation comprising the step of providing the solid oral dosage form of the present invention.

According to one aspect of the present invention, there is provided a pharmaceutical composition having a first component comprising active ingredient-containing particles, and at least one subsequent component comprising active ingredient-containing particles, each subsequent component having a rate and/or duration of release different from the first component wherein at least one of said components comprises loxoprofen-containing particles. The loxoprofen-containing particles may be coated with a modified release coating. Alternatively or additionally, the loxoprofen-containing particles may comprise a modified release matrix material. Following oral delivery, the composition delivers loxoprofen in a pulsatile manner. In one embodiment, the first component provides an immediate release of loxoprofen and the one or more subsequent components provide a modified release of loxoprofen. In such embodiments, the immediate release component serves to hasten the onset of action by minimizing the time from administration to a therapeutically effective plasma concentration level, and the one or more subsequent components serve to minimize the variation in plasma concentration levels and/or maintain a therapeutically effective plasma concentration throughout the dosing interval.

The modified release coating and/or the modified release matrix material cause a lag time between the release of the active ingredient from the first population of active ingredient-containing particles and the release of the active ingredient from subsequent populations of active ingredient-containing particles. Where more than one population of active ingredient-containing particles provide a modified release, the modified release coating and/or the modified release matrix material causes a lag time between the release of the active ingredient from the different populations of active ingredient-containing particles. The duration of these lag times may be varied by altering the composition and/or the amount of the modified release coating and/or altering the composition and/or amount of modified release matrix material utilized. Thus, the duration of the lag time can be designed to mimic a desired plasma profile.

Because the plasma profile produced by the modified release composition upon administration is substantially similar to the plasma profile produced by the administration of two or more IR dosage forms given sequentially, the modified release composition of the present invention is particularly useful for administering a loxoprofen which is normally administered three times daily. In one embodiment of the present invention, the composition delivers the loxoprofen in a trimodal manner. Upon administration, such a composition produces a plasma profile which substantially mimics that obtained by the sequential administration of three IR doses of loxoprofen in accordance with a typical treatment regimen.

According to another aspect of the present invention, the composition can be designed to produce a plasma profile that minimizes or eliminates the variations in plasma concentration levels associated with the administration of two or more IR dosage forms given sequentially. In such embodiments, the composition may be provided with an immediate release component to hasten the onset of action by minimizing the time from administration to a therapeutically effective plasma concentration level, and at least one modified release component to maintain a therapeutically effective plasma concentration level throughout the dosing interval.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules, or mixtures thereof, irrespective of their size, shape or morphology.

The term “modified release” as used herein includes a release which is not immediate and includes controlled release, extended release, sustained release and delayed release.

The term “time delay” as used herein refers to the period of time between the administration of a dosage form comprising the composition of the invention and the release of the active ingredient from a particular component thereof.

The term “lag time” as used herein refers to the time between the release of the active ingredient from one component of the composition and the release of the active ingredient from another component of the composition.

The term “erodable” as used herein refers to formulations which may be worn away, diminished, or deteriorated by the action of substances within the body.

The term “diffusion controlled” as used herein refers to formulations which may spread as the result of their spontaneous movement, for example, from a region of higher to one of lower concentration.

The term “osmotic controlled” as used herein refers to formulations which may spread as the result of their movement through a semi-permeable membrane into a solution of higher concentration that tends to equalize the concentrations of the formulation on the two sides of the membrane.

The active ingredients in each component may be the same or different. For example, the composition may comprise components comprising only loxoprofen as the active ingredient. Alternatively, the composition may comprise a first component comprising loxoprofen and at least one subsequent component comprising an active ingredient other than loxoprofen suitable for coadministration with loxoprofen, or a first component containing an active ingredient other than loxoprofen and at least one subsequent component comprising loxoprofen. Indeed, two or more active ingredients may be incorporated into the same component when the active ingredients are compatible with each other. An active ingredient present in one component of the composition may be accompanied by, for example, an enhancer compound or a sensitizer compound in another component of the composition, in order to modify the bioavailability or therapeutic effect thereof.

As used herein, the term “enhancer” refers to a compound which is capable of enhancing the absorption and/or bioavailability of an active ingredient by promoting net transport across the GIT in an animal, such as a human. Enhancers include but are not limited to medium chain fatty acids; salts, esters, ethers and derivatives thereof, including glycerides and triglycerides; non-ionic

surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

In those embodiments in which more than one loxoprofen-containing component is present, the proportion of loxoprofen contained in each component may be the same or different depending on the desired dosing regime. The loxoprofen present in the first component and in subsequent components may be any amount sufficient to produce a therapeutically effective plasma concentration level. The loxoprofen, when applicable, may be present either in the form of one substantially optically pure stereoisomer or as a mixture, racemic or otherwise, of two or more stereoisomers. The loxoprofen is preferably present in the composition in an amount of from about 0.1 to about 500 mg, preferably in the amount of from about 1 to about 100 mg. The loxoprofen is preferably present in the first component in an amount of from about 0.5 to about 60 mg; more preferably the loxoprofen, is present in the first component in an amount of from about 2.5 to about 30 mg. The loxoprofen is present in subsequent components in an amount within similar ranges to those described for the first component.

The time release characteristics for the delivery of the loxoprofen from each of the components may be varied by modifying the composition of each component, including modifying any of the excipients and/or coatings which may be present. In particular, the release of the loxoprofen may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time and/or time delay for the release of the loxoprofen from each component may also be varied by modifying the composition of each of the components, including modifying any excipients and coatings which may be present. For example, the first component may be an immediate release component wherein the loxoprofen is released immediately upon administration. Alternatively, the first component may be, for example, a time-delayed immediate release component in which the loxoprofen is released substantially in its entirety immediately after a time delay. The second and subsequent component may be, for example, a time-delayed immediate release component as just described or, alternatively, a time-delayed sustained release or extended release component in which the loxoprofen is released in a controlled fashion over an extended period of time.

As will be appreciated by those skilled in the art, the exact nature of the plasma concentration curve will be influenced by the combination of all of these factors just described. In particular, the lag time between the delivery (and thus also the onset of action) of the loxoprofen in each component may be controlled by varying the composition and coating (if present) of each of the components. Thus by variation of the composition of each component (including the amount and nature of the active ingredient(s)) and by variation of the lag time, numerous release and plasma profiles may be obtained. Depending on the duration of the lag time between the release of the loxoprofen from each component and the nature of the release of the loxoprofen from each component (i.e. immediate release, sustained release etc.), the plasma profile may be continuous (i.e., having a single maximum) or pulsatile in which the peaks in the plasma profile may be well separated and clearly defined (e.g. when the lag time is long) or superimposed to a degree (e.g. when the lag time is short).

The plasma profile produced from the administration of a single dosage unit comprising the composition of the present invention is advantageous when it is desirable to deliver two or more pulses of active ingredient without the need for administration of two or more dosage units. Additionally, in the case of treating pain and/or inflammation, it is particularly useful to have such a multimodal plasma profile. For example, a typical loxoprofen treatment regime consists of the administration of two or three doses of an immediate release dosage formulation given four hours apart. This type of regime has been found to be therapeutically effective and is widely used.

Any coating material which modifies the release of the loxoprofen in the desired manner may be used. In particular, coating materials suitable for use in the practice of the present invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the trademark Eudragit® RS and RL, poly acrylic acid and poly acrylate and methacrylate copolymers such as those sold under the trademark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, polyvinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (mol. wt. ˜5 k-5,000 k), polyvinylpyrrolidone (mol. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (mol. wt. ˜30 k-300 k), polysaccharides such as agar, acacia, karaya, tragacanth, algins and guar, polyacrylamides, Polyox® polyethylene oxides (mol. wt. ˜100 k-5,000 k), AquaKeep® acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of a loxoprofen dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

A modified release composition according to the present invention may be incorporated into any suitable dosage form which facilitates release of the active ingredient in a pulsatile manner. In one embodiment, the dosage form comprises a blend of different populations of active ingredient-containing particles which make up the immediate release and the modified release components, the blend being filled into suitable capsules, such as hard or soft gelatin capsules. Alternatively, the different individual populations of active ingredient-containing particles may be compressed (optionally with additional excipients) into mini-tablets which may be subsequently filled into capsules in the appropriate proportions. Another suitable dosage form is that of a multilayer tablet. In this instance the first component of the modified release composition may be compressed into one layer, with the second component being subsequently added as a second layer of the multilayer tablet. The populations of loxoprofen-containing particles making up the composition of the invention may further be included in rapidly dissolving dosage forms such as an effervescent dosage form or a fast-melt dosage form.

In one embodiment, the composition comprises at least two loxoprofen components: a first loxoprofen component and one or more subsequent loxoprofen components. In such embodiment, the first loxoprofen component of the composition may exhibit a variety of release profiles including profiles in which substantially all of the loxoprofen contained in the first component is released rapidly upon administration of the dosage form, released rapidly but after a time delay (delayed release), or released slowly over time. In one such embodiment, the loxoprofen contained in the first component is released rapidly upon administration to a patient. As used herein, “released rapidly” includes release profiles in which at least about 80% of the active ingredient of a component is released within about an hour after administration, the term “delayed release” includes release profiles in which the active ingredient of a component is released (rapidly or slowly) after a time delay, and the terms “controlled release” and “extended release” include release profiles in which at least about 80% of the active ingredient contained in a component is released slowly.

The second loxoprofen component of such embodiment may also exhibit a variety of release profiles including an immediate release profile, a delayed release profile or a controlled release profile. In one such embodiment, the second loxoprofen component exhibits a delayed release profile in which the loxoprofen of the component is released after a time delay. In another such embodiment, the second loxoprofen component exhibits a controlled release profile in which the loxoprofen of the component is released over a period of about 12 to about 24 hours after administration.

In embodiments in which the first loxoprofen component exhibits an immediate release profile and the second loxoprofen component exhibits a modified release profile, the loxoprofen in the first and second components are released over different time periods. In one such embodiment, the loxoprofen in the first component is released rapidly and the loxoprofen in the second component is released within a period of about 12 hours after administration. In another such embodiment, the loxoprofen in the first component is released rapidly and the loxoprofen in the second component is released within a period of about 24 hours after administration. In yet another such embodiment, the loxoprofen in the first component is released rapidly and the loxoprofen in the second component is released over a period of about 12 hours after administration. In still another such embodiment, the loxoprofen in the first loxoprofen component is released rapidly and the loxoprofen in the second loxoprofen component is released over a period of about 24 hours after administration. In yet another such embodiment, the loxoprofen in the first loxoprofen component is released rapidly and the loxoprofen in the second loxoprofen component is released over a period of at least about 12 hours after administration. In still another such embodiment, the loxoprofen in the first loxoprofen component is released rapidly and the loxoprofen in the second loxoprofen component is released over a period of at least about 24 hours after administration.

The plasma profile produced by the administration of dosage forms of the present invention which comprise an immediate release loxoprofen component and at least one modified release loxoprofen component can be substantially similar to the plasma profile produced by the administration of two or more IR dosage forms given sequentially, or to the plasma profile produced by the administration of separate IR and modified release dosage forms. Accordingly, the dosage forms of the present invention can be particularly useful for administering loxoprofen where the maintenance of pharmacokinetic parameters may be desired but is problematic.

In one embodiment, the composition and the solid oral dosage forms containing the composition release the loxoprofen such that substantially all of the loxoprofen contained in the first component is released prior to release of the loxoprofen from the at least one second component. When the first component comprises an IR component, for example, it is preferable that release of the loxoprofen from the at least one second component is delayed until substantially all the loxoprofen in the IR component has been released. Release of the loxoprofen from the at least one second component may be delayed as detailed above by the use of a modified release coatings and/or a modified release matrix material.

When it is desirable to minimize patient tolerance by providing a dosage regime which facilitates wash-out of a first dose of the loxoprofen from a patient's system, release of the loxoprofen from subsequent components may be delayed until substantially all of the loxoprofen contained in the first component has been released, and further delayed until at least a portion the loxoprofen released from the first component has been cleared from the patient's system. In one embodiment, release of the loxoprofen from subsequent components of the composition is substantially, if not completely, delayed for a period of at least about two hours after administration of the composition. In another embodiment, the release of loxoprofen from subsequent components of the composition is substantially, if not completely, delayed for a period of at least about four hours, preferably about four hours, after administration of the composition.

As described hereinbelow, the present invention also includes various types of modified release systems by which loxoprofen may be delivered in either a pulsatile or continuous manner. These systems include but are not limited to: films with loxoprofen in a polymer matrix (monolithic devices); loxoprofen contained by the polymer (reservoir devices); polymeric colloidal particles or microencapsulates (microparticles, microspheres or nanoparticles) in the form of reservoir and matrix devices; loxoprofen contained by a polymer containing a hydrophilic and/or leachable additive e.g., a second polymer, surfactant or plasticizer, etc. to give a porous device, or a device in which loxoprofen release may be osmotically controlled (both reservoir and matrix devices); enteric coatings (ionizable and dissolve at a suitable pH); (soluble) polymers with (covalently) attached pendant drug molecules; and devices where release rate is controlled dynamically: e.g., the osmotic pump.

The delivery mechanism of the present invention can control the rate of release of loxoprofen. While some mechanisms will release loxoprofen at a constant rate, others will vary as a function of time depending on factors such as changing concentration gradients or additive leaching leading to porosity, etc.

Polymers used in sustained release coatings are necessarily biocompatible, and ideally biodegradable. Examples of both naturally occurring polymers such as Aquacoat® (FMC Corporation, Food & Pharmaceutical Products Division, Philadelphia, USA) (ethylcellulose mechanically spheronised to sub-micron sized, aqueous based, pseudo-latex dispersions), and also synthetic polymers such as the Eudragit® (Röhm Pharma, Weiterstadt.) range of poly(acrylate, methacrylate) copolymers are known in the art.

Reservoir Devices

A typical approach to modified release is to encapsulate or contain the drug entirely (e.g., as a core), within a polymer film or coat (i.e., microcapsules or spray/pan coated cores).

The various factors that can affect the diffusion process may readily be applied to reservoir devices (e.g., the effects of additives, polymer functionality (and, hence, sink-solution pH) porosity, film casting conditions, etc.) and, hence, the choice of polymer must be an important consideration in the development of reservoir devices. Modeling the release characteristics of reservoir devices (and monolithic devices) in which the transport of the drug is by a solution-diffusion mechanism therefore typically involves a solution to Fick's second law (unsteady-state conditions; concentration dependent flux) for the relevant boundary conditions. When the device contains dissolved active agent, the rate of release decreases exponentially with time as the concentration (activity) of the agent (i.e., the driving force for release) within the device decreases (i.e., first order release). If, however, the active agent is in a saturated suspension, then the driving force for release is kept constant until the device is no longer saturated. Alternatively the release-rate kinetics may be desorption controlled, and a function of the square root of time.

Transport properties of coated tablets, may be enhanced compared to free-polymer films, due to the enclosed nature of the tablet core (permeant) which may enable the internal build-up of an osmotic pressure which will then act to force the permeant out of the tablet.

The effect of de-ionized water on salt containing tablets coated in poly(ethylene glycol) (PEG)-containing silicone elastomer, and also the effects of water on free films has been investigated. The release of salt from the tablets was found to be a mixture of diffusion through water filled pores, formed by hydration of the coating, and osmotic pumping. KCl transport through films containing just 10% PEG was negligible, despite extensive swelling observed in similar free films, indicating that porosity was necessary for the release of the KCl which then occurred by trans-pore diffusion. Coated salt tablets, shaped as disks, were found to swell in de-ionized water and change shape to an oblate spheroid as a result of the build-up of internal hydrostatic pressure: the change in shape providing a means to measure the force generated. As might be expected, the osmotic force decreased with increasing levels of PEG content. The lower PEG levels allowed water to be imbibed through the hydrated polymer, while the porosity resulting from the coating dissolving at higher levels of PEG content (20 to 40%) allow the pressure to be relieved by the flow of KCl.

Methods and equations have been developed, which by monitoring (independently) the release of two different salts (e.g., KCl and NaCl) allowed the calculation of the relative magnitudes that both osmotic pumping and trans-pore diffusion contributed to the release of salt from the tablet. At low PEG levels, osmotic flow was increased to a greater extent than was trans-pore diffusion due to the generation of only a low pore number density: at a loading of 20%, both mechanisms contributed approximately equally to the release. The build-up of hydrostatic pressure, however, decreased the osmotic inflow, and osmotic pumping. At higher loadings of PEG, the hydrated film was more porous and less resistant to outflow of salt. Hence, although the osmotic pumping increased (compared to the lower loading), trans-pore diffusion was the dominant release mechanism. An osmotic release mechanism has also been reported for microcapsules containing a water soluble core.

Monolithic Devices (Matrix Devices)

Monolithic (matrix) devices are commonly used for controlling the release of drugs. This is possibly because they are relatively easy to fabricate compared to reservoir devices, and the danger of an accidental high dosage that could result from the rupture of the membrane of a reservoir device is not present. In such a device, the active agent is present as a dispersion within the polymer matrix, and they are typically formed by the compression of a polymer/drug mixture or by dissolution or melting. The dosage release properties of monolithic devices may be dependent upon the solubility of the drug in the polymer matrix or, in the case of porous matrixes, the solubility in the sink solution within the particle's pore network, and also the tortuosity of the network (to a greater extent than the permeability of the film), dependent on whether the drug is dispersed in the polymer or dissolved in the polymer. For low loadings of drug, (0 to 5% W/V) the drug will be released by a solution-diffusion mechanism (in the absence of pores). At higher loadings (5 to 10% W/V), the release mechanism will be complicated by the presence of cavities formed near the surface of the device as the drug is lost: such cavities fill with fluid from the environment increasing the rate of release of the drug.

It is common to add a plasticizer (e.g., a poly(ethylene glycol)), a surfactant, or adjuvant (i.e., an ingredient which increases effectiveness), to matrix devices (and reservoir devices) as a means to enhance the permeability (although, in contrast, plasticizers may be fugitive, and simply serve to aid film formation and, hence, decrease permeability—a property normally more desirable in polymer paint coatings). It was noted that the leaching of PEG increased the permeability of (ethyl cellulose) films linearly as a function of PEG loading by increasing the porosity, however, the films retained their barrier properties, not permitting the transport of electrolyte. It was deduced that the enhancement of their permeability was as a result of the effective decrease in thickness caused by the PEG leaching. This was evidenced from plots of the cumulative permeant flux per unit area as a function of time and film reciprocal thickness at a PEG loading of 50% W/W: plots showing a linear relationship between the rate of permeation and reciprocal film thickness, as expected for a (Fickian) solution-diffusion type transport mechanism in a homogeneous membrane. Extrapolation of the linear regions of the graphs to the time axis gave positive intercepts on the time axis: the magnitude of which decreased towards zero with decreasing film thickness. These changing lag times were attributed to the occurrence of two diffusional flows during the early stages of the experiment (the flow of the drug and also the flow of the PEG), and also to the more usual lag time during which the concentration of permeant in the film is building-up. Caffeine, when used as a permeant, showed negative lag times. No explanation of this was forthcoming, but it was noted that caffeine exhibited a low partition coefficient in the system, and that this was also a feature of aniline permeation through polyethylene films which showed a similar negative time lag.

The effects of added surfactants on (hydrophobic) matrix devices has been investigated. It was thought that surfactant may increase the drug release rate by three possible mechanisms: (i) increased solubilization, (ii) improved wettability to the dissolution media, and (iii) pore formation as a result of surfactant leaching. For the system studied (Eudragit® RL 100 and RS 100 plasticised by sorbitol, flurbiprofen as the drug, and a range of surfactants) it was concluded that improved wetting of the tablet led to only a partial improvement in drug release (implying that the release was diffusion, rather than dissolution, controlled), although the effect was greater for Eudragit® RS than Eudragit® RL, while the greatest influence on release was by those surfactants that were more soluble due to the formation of disruptions in the matrix allowing the dissolution medium access to within the matrix. This is of obvious relevance to a study of latex films which might be suitable for pharmaceutical coatings, due to the ease with which a polymer latex may be prepared with surfactant as opposed to surfactant-free. Differences were found between the two polymers with only the Eudragit® RS showing interactions between the anionic/cationic surfactant and drug. This was ascribed to the differing levels of quaternary ammonium ions on the polymer.

Composite devices consisting of a polymer/drug matrix coated in a polymer containing no drug also exist. Such a device was constructed from aqueous Eudragit® lattices, and was found to provide a continuous release by diffusion of the drug from the core through the shell. Similarly, a polymer core containing the drug has been produced and coated with a shell that was eroded by gastric fluid. The rate of release of the drug was found to be relatively linear (a function of the rate limiting diffusion process through the shell) and inversely proportional to the shell thickness, whereas the release from the core alone was found to decrease with time.

Microspheres

Methods for the preparation of hollow microspheres have been described. Hollow microspheres were formed by preparing a solution of ethanol/dichloromethane containing the drug and polymer. On pouring into water, an emulsion is formed containing the dispersed polymer/drug/solvent particles, by a coacervation-type process from which the ethanol rapidly diffused precipitating polymer at the surface of the droplet to give a hard-shelled particle enclosing the drug dissolved in the dichloromethane. A gas phase of dichloromethane was then generated within the particle which, after diffusing through the shell, was observed to bubble to the surface of the aqueous phase. The hollow sphere, at reduced pressure, then filled with water which could be removed by a period of drying. No drug was found in the water. Highly porous matrix-type microspheres have also been described. The matrix-type microspheres were prepared by dissolving the drug and polymer in ethanol. On addition to water, the ethanol diffused from the emulsion droplets to leave a highly porous particle. A suggested use of the microspheres was as floating drug delivery devices for use in the stomach.

Pendent Devices

A means of attaching a range of drugs such as analgesics and antidepressants, etc., by means of an ester linkage to poly(acrylate) ester latex particles prepared by aqueous emulsion polymerization has been developed. These lattices, when passed through an ion exchange resin such that the polymer end groups were converted to their strong acid form, could self-catalyze the release of the drug by hydrolysis of the ester link.

Drugs have been attached to polymers, and also monomers have been synthesized with a pendent drug attached. Dosage forms have been prepared in which the drug is bound to a biocompatible polymer by a labile chemical bond e.g., polyanhydrides prepared from a substituted anhydride (itself prepared by reacting an acid chloride with the drug: methacryloyl chloride and the sodium salt of methoxy benzoic acid) were used to form a matrix with a second polymer (Eudragit® RL) which released the drug on hydrolysis in gastric fluid. The use of polymeric Schiff bases suitable for use as carriers of pharmaceutical amines has also been described.

Enteric Films

Enteric coatings consist of pH sensitive polymers. Typically the polymers are carboxylated and interact very little with water at low pH, while at high pH the polymers ionize causing swelling or dissolving of the polymer. Coatings can therefore be designed to remain intact in the acidic environment of the stomach, protecting either the drug from this environment or the stomach from the drug, but to dissolve in the more alkaline environment of the intestine.

Osmotically Controlled Devices

The osmotic pump is similar to a reservoir device but contains an osmotic agent (e.g., the active agent in salt form) which acts to imbibe water from the surrounding medium via a semi-permeable membrane. Such a device, called an elementary osmotic pump, has been described. Pressure is generated within the device which forces the active agent out of the device via an orifice of a size designed to minimize solute diffusion, while preventing the build-up of a hydrostatic pressure head which can have the effect of decreasing the osmotic pressure and changing the dimensions of the device. While the internal volume of the device remains constant, and there is an excess of solid or saturated solution in the device, then the release rate remains constant delivering a volume equal to the volume of solvent uptake.

Electrically Stimulated Release Devices

Monolithic devices have been prepared using polyelectrolyte gels which swell when, for example, an external electrical stimulus is applied causing a change in pH. The release may be modulated by changes in the applied current to produce a constant or pulsatile release profile.

Hydrogels

In addition to their use in drug matrices, hydrogels find use in a number of biomedical applications such as, for example, soft contact lenses, and various soft implants, and the like.

### Methods of Using Modified Release Loxoprofen Compositions

According to another aspect of the present invention, there is provided a method for treating a patient suffering from pain and/or inflammation comprising the step of administering a therapeutically effective amount of the loxoprofen composition of the present invention in solid oral dosage form. Advantages of the method of the present invention include a reduction in the dosing frequency required by conventional multiple IR dosage regimes while still maintaining the benefits derived from a pulsatile plasma profile or eliminating or minimizing the variations in plasma concentration levels. This reduced dosing frequency is advantageous in terms of patient compliance and the reduction in dosage frequency made possible by the method of the present invention would contribute to controlling health care costs by reducing the amount of time spent by health care workers on the administration of drugs.

In the following examples, all percentages are weight by weight unless otherwise stated. The term “purified water” as used throughout the Examples refers to water that has been purified by passing it through a water filtration system. It is to be understood that the examples are for illustrative purposes only, and should not be interpreted as restricting the spirit and breadth of the invention as defined by the scope of the claims that follow.

### Example 1

### Multiparticulate Modified Release Composition Containing Loxoprofen

A multiparticulate modified release composition according to the present invention comprising an immediate release component and a modified release component containing loxoprofen is prepared as follows.

### (a) Immediate Release Component.

A solution of loxoprofen is prepared according to any of the formulations given in Table 1. The loxoprofen solution is then coated onto nonpareil seeds to a level of approximately 16.9% solids weight gain using, for example, a Glatt GPCG3 (Glatt, Protech Ltd., Leicester, UK) fluid bed coating apparatus to form the IR particles of the immediate release component.

TABLE 1
Immediate release component solutions

| | Amount (% (w/w)) | Amount (% (w/w)) |
|---|---|---|
| Ingredient | (i) | (ii) |
| Loxoprofen | 13.0 | 13.0 |
| Polyethylene Glycol 6 | 0.5 | 0.5 |
| Polyvinylpyrrolidone | 3.5 | |
| Purified Water | 83.5 | 86.5 |

### (b) Modified Release Component

Loxoprofen containing delayed release particles are prepared by coating immediate release particles prepared according to Example 1 (a) above with a modified release coating solution as detailed in Table 2. The immediate release particles are coated to varying levels up to approximately to 30% weight gain using, for example, a fluid bed apparatus.

TABLE 2
Modified release component coating solutions
Amount, % (w/w)

| Ingredient | (i) | (ii) | (iii) | (iv) | (v) | (vi) | (vii) | (viii) |
|---|---|---|---|---|---|---|---|---|
| Eudragit ® | 49.7 | 42.0 | 47.1 | 53.2 | 40.6 | — | — | 25.0 |
| RS 12.5 | | | | | | | | |
| Eudragit ® | — | — | — | — | — | 54.35 | 46.5 | — |
| S 12.5 | | | | | | | | |
| Polyvinyl- | — | — | — | 0.35 | 0.3 | — | — | — |
| pyrrolidone | | | | | | | | |
| Diethyl- | 0.5 | 0.5 | 0.6 | 1.35 | 0.6 | 1.3 | 1.1 | — |
| phthalate | | | | | | | | |
| Triethyl- | — | — | — | — | — | — | — | 1.25 |
| citrate | | | | | | | | |
| Isopropyl | 39.8 | 33.1 | 37.2 | 45.1 | 33.8 | 44.35 | 49.6 | 46.5 |
| alcohol | | | | | | | | |
| Acetone | 10.0 | 8.3 | 9.3 | — | 8.4 | — | — | — |
| Talc¹ | — | 16.0 | 5.9 | — | 16.3 | — | 2.8 | 2.25 |

¹Talc is simultaneously applied during coating for formulations in column (i), (iv) and (vi).

### (c) Encapsulation of Immediate and Delayed Release Particles.

The immediate and delayed release particles prepared according to Example 1(a) and (b) above are encapsulated in size 2 hard gelatin capsules to an overall 20 mg dosage strength using, for example, a Bosch GKF 4000S encapsulation apparatus. The overall dosage strength of 20 mg loxoprofen was made up of 10 mg from the immediate release component and 10 mg from the modified release component.

### Example 2

### Multiparticulate Modified Release Composition Containing Loxoprofen

Multiparticulate modified release loxoprofen compositions according to the present invention having an immediate release component and a modified release component having a modified release matrix material are prepared according to the formulations shown in Table 5(a) and (b).

TABLE 5 (a)
100 mg of IR component is encapsulated with 100 mg of modified
release (MR) component to give a 20 mg dosage strength product
% (w/w)

| IR component | |
|---|---|
| Loxoprofen | 10 |
| Microcrytalline cellulose | 40 |
| Lactose | 45 |
| Povidone | 5 |
| MR component | |
| Loxoprofen | 10 |
| Microcrytalline cellulose | 40 |
| Eudragit ® RS | 45 |
| Povidone | 5 |

TABLE 5 (b)
50 mg of IR component is encapsulated with 50 mg of modified
release (MR) component to give a 20 mg dosage strength product.
% (w/w)

| IR component | |
|---|---|
| Loxoprofen | 20 |
| Microcrytalline cellulose | 50 |
| Lactose | 28 |
| Povidone | 2 |
| MR component | |
| Loxoprofen | 20 |
| Microcrytalline cellulose | 50 |
| Eudragit ® RS | 28 |
| Povidone | 2 |

### Example 3

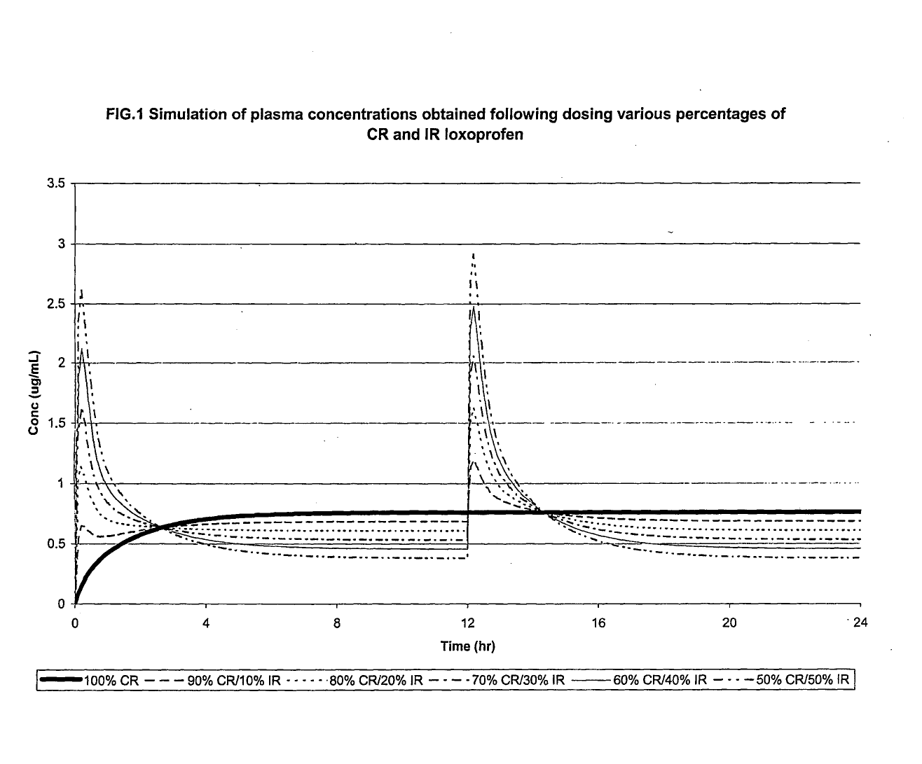

Simulations demonstrate that a modified release (CR) formulation using a pulsatile release approach can be developed that would improve patient convenience, enhance efficacy and improve safety. FIG. 1 is a graphical representation of a simulation of plasma concentrations obtained following dosing various percentages of modified release (CR) and immediate release (IR) loxoprofen. A 100% CR formulation increases gradually and then stabilizes at a plasma concentration of about 0.75 μg/ml while at the other extreme a 50% CR/50% IR formulation has peaks of over 2.5 μg/ml at 0 and 12 hours.

The pulsatile system can minimize the variation in plasma concentration levels exhibited by administration of immediate-release dosage forms resulting in more consistent blood levels and improved efficacy. The pulsatile release formulation also minimize GI irritation by decreasing incidences of locally high concentrations of the NSAID. Loxoprofen is currently administered three times daily. The modified release formulation can be administered twice a day thus improving patient compliance.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

1. A pharmaceutical composition comprising a first component of active ingredient-containing particles and at least one subsequent component of active ingredient-containing particles, wherein at least one of said components comprises loxoprofen and at least one of said components further comprises a modified release coating, a modified release matrix material, or both, such that said composition delivers the active ingredient containing particles to a patient in a pulsatile manner upon administration of said composition to said patient.

2. A pharmaceutical composition comprising a first component of active ingredient-containing particles and at least one subsequent component of active ingredient-containing particles, wherein at least one of said components comprises loxoprofen and at least one of said components further comprises a modified release coating, a modified release matrix material, or both, such that said composition delivers the active ingredient containing particles to a patient in a continuous manner upon administration of said composition to said patient.

3. The composition of claim 1 wherein each component comprises loxoprofen-containing particles.

4. The composition of claim 3 wherein the composition comprises a first component of loxoprofen-containing particles and one subsequent component of loxoprofen-containing particles.

5. The composition of claim 4, wherein the first component comprises an immediate release component and the second component comprises a modified release component.

6. The composition of claim 1, wherein the active ingredient-containing particles are erodable.

7. The composition of claim 1, wherein the active ingredient-containing particles have a modified-release coating.

8. The composition of claim 1, wherein the active ingredient-containing particles comprise a modified-release matrix material.

9. The composition of claim 1 wherein said modified release material is selected from the group consisting of hydrophilic polymers, hydrophobic polymers, natural polymers, synthetic polymers and mixtures thereof

10. The composition of claim 9 wherein the loxoprofen is released to the surrounding environment by erosion.

11. The composition of claim 10 wherein said composition further comprises an enhancer.

12. The composition according to claim 10 wherein the amount of loxoprofen contained therein is from about 0.1 mg to about 1 g.

13. A dosage form comprising the composition of claim 1.

14. The dosage form of claim 13 comprising a blend of active ingredient-containing particles contained within a hard gelatin or soft gelatin capsule.

15. The dosage form of claim 14, wherein the active ingredient-containing particles are in the form of mini-tablets and the capsule contains a mixture of said mini-tablets.

16. The dosage form of claim 15 in the form of tablet.

17. The dosage form of claim 16 wherein the loxoprofen-containing particles are provided in a rapidly dissolving dosage form.

18. The dosage form according to claim 16 wherein the tablet is a fast-melt tablet.

19. A method for the treatment of inflammation comprising the step of administering a therapeutically effective amount of the composition of claim 1.

20. The method of claim 19, wherein said inflammation is associated with a condition selected from the group consisting of a muscle disorder, a skeletal disorder, a joint disorder, an operative procedure, chronic arthritis, rheumatism, lumbago, and trauma.

21. The composition of claim 1 wherein the modified-release coating comprises a pH-dependent polymer coating for releasing a pulse of the active ingredient in said patient following a time delay of about 6 to about 12 hours after administration of said composition to said patient.

22. The composition according to claim 21, wherein said polymer coating comprises methacrylate copolymers.

23. The composition according to claim 21, wherein the polymer coating comprises a mixture of methacrylate and ammonio methacrylate copolymers in a ratio sufficient to achieve a pulse of the active ingredient following a time delay of at least about 6 hours.

24. The composition according to claim 23, wherein the ratio of methacrylate to ammonio methacrylate copolymers is approximately 1:1.
